(19) United States
(12) Patent Application Publication
(10) Pub. No.: US 2011/0212072 A1
(43) Pub. Date: Sep. 1, 2011

(54) Inhibitors of Fatty Acid Oxidation for Prophylaxis and Treatment of Diseases Related to Mitochondrial Dysfunction

(75) Inventors: Thomas Henkel, Kassel (DE); Christoph Rehfuess, Munich (DE)

(73) Assignee: MediGene AG, Martinsried (DE)

(21) Appl. No.: 13/089,979

(22) Filed: Apr. 19, 2011

Related U.S. Application Data
(63) Continuation of application No. 12/459,227, filed on Jun. 29, 2009, now abandoned.
Application No. 12/459,227 is a continuation of application No. 10/832,801, filed on Apr. 26, 2004, now abandoned.
Application No. 10/832,801 is a continuation of application No. PCT/EP2002/011913, filed on Oct. 24, 2002.
(60) Provisional application No. 60/380,269, filed on May 15, 2002.
(60) Provisional application No. 60/330,647, filed on Oct. 26, 2001.

(51) Int. Cl. A61K 38/44; A61K 31/7088; A61K 38/46; A61K 38/51; A61K 38/43; A61P 25/28; A61P 25/16; A61P 29/00; A61P 9/10; A61P 25/18; A61P 25/08; A61P 25/06; A61P 5/18; C12Q 1/02; C12Q 1/37; G01N 33/53; A61K 31/4458; C07D 303/40; A61K 31/336; C12Q 1/68; C07D 211/12; C07D 295/096; A61K 31/495; C07C 229/34; A61K 31/198; C07C 57/03; A61K 31/202; C07C 309/47; A61K 31/185

(52) U.S. Cl. 424/94.4; 514/44 A; 424/94.6; 424/94.5; 424/94.1; 514/44 R; 435/29; 435/23; 435/7.1; 514/317; 549/549; 514/475; 435/6.1; 546/192; 544/398; 514/252.12; 562/444; 514/561; 554/224; 514/560; 562/63; 514/577

(57) ABSTRACT

Described are a method and a composition for preventing and/or treating a disease related to mitochondrial dysfunction by inhibiting the fatty acid oxidation of one or more cells of an organism. Particularly, the fatty acid oxidation is inhibited by inhibiting the expression and/or activity of the enzyme Camitin-Palmitoyl-Transferase-1 (CPT-1) by means of an arylalkyl- or arlyoxyalkyl-substitued oxirane carboxylic acid or pharmaceutically acceptable salts and derivatives of the arylalkyl-substituted oxirane carboxylic acid.

The present invention refers to the identification of therapeutic methods and pharmaceutical compositions for the prophylaxis and/or treatment of diseases related to mitochondrial dysfunction by inhibiting the oxidation of fatty acids.

Mitochondria are the organdies of eukaryotic cells which can be denoted as the “power to sources” of these cells. In the mitochondria, the cellular respiration takes place. Cellular respiration comprises the process by which organic molecules are broken down to release energy in the form of high-energy phosphate bonds of adenosine uiphosphate (ATP), the most important source of energy for an organism.

Thus, mitochondrial energy production is a basis for physical condition and health. The consequences of deficient mitochondria can be destructive at the levels of cells, tissues, and organisms. Mitochondrial dysfunction is implicated in the pathogenesis of, for example, degenerative diseases such as Morbus Alzheimer, Morbus Huntington, Morbus Parkinson, amyotrophic lateral sclerosis, inflammatory diseases, furthermore of acute traumatic events, such as surgery or injury, moreover of AIDS related wasting due to the toxicity of reverse transcriptase inhibitors, of mitochondrial myopathies and of senescence and ageing.

In the United States, more than 50 million adults suffer from diseases in which mitochondrial dysfunction is involved and it is estimated that of the 4 million children born in the U.S. each year, up to 4000 develop mitochondrial diseases. To date, there is no therapy for mitochondrial diseases.

Accordingly, it is the problem of the present invention to provide therapeutic approaches and pharmaceutical compositions which are useful in the prevention and/or treatment of diseases related to mitochondrial dysfunction.

The present invention is based on the unexpected finding that the inhibition of the oxidation of fatty acids elicits a positive effect on cellular function due to the reduction of oxidative stress to mitochondria.

Mitochondrial fatty acid oxidation (FAO) is a potent source of reactive oxygen intermediates such as peroxides. Increased production of said reactive oxygen intermediates is generally regarded as a pro-inflammatory and degenerative event. Especially in situations of oxygenation/re-oxygenation and limited substrate oxidation, increased β-oxidation without sufficient end-oxidation via the respiratory complexes I to IV can result in severe impairment of oxidative phosphorylation and mitochondrial dysfunction.

Consequently, the production of reactive oxygen intermediates can be reduced if the to mitochondrial fatty acid oxidation is inhibited. One important advantage of the present invention is the efficacy of the methods and compounds used independent from the cause and progression of the mitochondrial dysfunction. Hence, the present invention is concerned with preventing or treating a disease related to mitochondrial dysfunction by inhibiting the fatty acid oxidation in mitochondria. The organism to be treated with the methods of the present invention is preferably a human being.

In the context of the present invention, the term “mitochondrial dysfunction” means any kind of impaired function of mitochondria which can be inherited, sporadic or induced by the environment.

As used herein, the terms “inhibitor” or “inhibiting agent” refer to any compound capable of down-regulating, decreasing, reducing, suppressing, inactivating or otherwise regulating the amount and/or activity of an enzyme, particularly the enzymes involved in fatty acid oxidation referred to below. Generally, these inhibitors or inhibiting agents may be proteins, oligo- and polypeptides, nucleic acids, genes, and chemical molecules. Suitable protein inhibitors may be, for example, monoclonal or polyclonal antibodies which bind to one of the enzymes described below. Inhibition of enzymes can be achieved by any of a variety of mechanisms known in the art, including, but not limited to, binding directly to the enzyme (e.g., enzyme inhibitor compound binding complex or substrate mimetic), denaturing or otherwise inactivating the enzyme, inhibiting the expression of a gene which encodes the enzyme (e.g., transcription to mRNA, translation to a nascent polypeptide) and/or final modifications to a mature protein.

As used herein, the term “regulating” or “regulation” refers to the ability of an inhibitor to down-regulate, decrease, reduce, suppress, or inactivate at least partially the activity and/or expression of an enzyme. As used herein, the term “regulating the expression and/or activity” generally refers to any process that functions to control or modulate the quantity or activity (functionality) of a cellular component, particularly an enzyme. Static regulation maintains expression and/or activity at some given level. Up-regulation refers to a relative increase in expression and/or activity, Accordingly, down-regulation refers to a decrease in expression and/or activity. According to the present invention, regulation is preferably the down-regulation of a cellular component, especially an enzyme. Down-regulation is synonymous to with the inhibition of a given cellular component's expression and/or activity.

As used herein, a “pharmaceutically effective amount” of an inhibitor is an amount effective to achieve the desired physiological result, either in cells treated in vitro or in a subject treated in vivo. Specifically, a pharmaceutically effective amount is an amount sufficient to inhibit, for some period of time, one or more clinically defined pathological effects associated with the mitochondrial dysfunction. The pharmaceutically effective amount may vary depending on the specific inhibitor selected, and is also dependent on a variety of factors and conditions related to the subject to be treated and the severity of the disease. For example, if the inhibitor is to be administered in vivo, factors such as age, weight, sex, and general health of the patient as well as dose response curves and toxicity data obtained in pre-clinical animal tests would be among the factors to be considered. If the inhibitor is to be contacted with cells in vitro, one would also design a variety of pre-clinical in vitro studies to assess parameters like uptake, half-life, dose, toxicity etc. The determination of a pharmaceutically effective amount for a given agent (inhibitor) is well within the ability of those skilled in the art.

Based on the findings of the inventors, one aspect of the present invention is directed to a method for regulating the fatty acid oxidation in mitochondria in an organism, particularly a human being, by administering to the organism a pharmaceutically effective amount of an inhibitor which inhibits at least partially the activity of one or more enzymes of the fatty acid oxidation in mitochondria.

According to the present invention, compounds can be identified which are useful for prophylaxis and/or treatment of mitochondrial dysfunction and the diseases related therewith. Suitable compounds can be identified by screening test compounds, or a library of test compounds, for their ability to inhibit the fatty acid oxidation of cells, particularly to inhibit enzymes of the fatty acid oxidation of cells. The compound(s) that has/have proven to be effective in inhibiting the fatty acid oxidation can then be used for manufacturing a pharmaceutical composition for the prophylaxis and/or treatment of mitochondrial dysfunction and the diseases related therewith. Accordingly, another aspect of the present invention is directed to a therapeutic composition useful to treat an individual afflicted with mitochondrial dysfunction and the diseases related therewith, respectively.

According to a preferred embodiment of the present invention, the fatty acid oxidation of to mitochondria can be reduced by the inhibition of the expression and/or activity of the enzyme Carnitin-Palmitoyl-Transferase-1 (CPT-1), which is the key enzyme of the fatty acid oxidation. Potent inhibitors of CPT-1 and thus mitochondrial fatty acid oxidation are arylalkyl- and aryloxyalkyl-substituted oxirane carboxylic acids of the following formula I

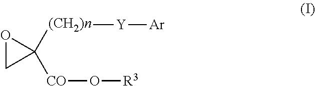

wherein
Ar is a substituted phenyl radical

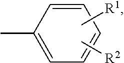

a 1- or 2-naphthyl radical which is substituted by a radical R⁴, or a heterocyclic radical Het;
R¹ is a hydrogen atom, a halogen atom, a 1-4 C lower alkyl group, a 1-4 C lower alkoxy group, a nitro group, or a trifluoromethyl group;
R² is one of the groups

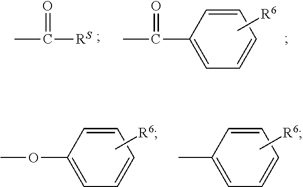

or a fully or predominantly fluorine-substituted 1-3 C alkoxy group or has one of the meanings of R¹;
R³ is a hydrogen atom or a 1-4 C lower alkyl group;
R⁴ e is a hydrogen atom, a 1-4 C lower alkyl group, an optionally fully or predominantly fluorine-substituted 1-3 C alkoxy group, or a halogen atom;
R⁵ is a 1-4 C lower alkyl group;
R⁶ is a hydrogen atom, a halogen atom, or a 1-4 C lower alkyl group;
Y is the grouping —O— or —CH₂—;

n is an integer from 2 to 8; and
Het is a heterocyclic ring, which preferably has 5 members and is selected from the group consisting of thiophene, thiazole, isothiazole, pyrrole, and, particularly preferably, pyrazole, and which may carry 1 or 2 identical or different substituents R¹;
   whereby the chain —(CH₂)— may optionally be interrupted by a —CH(CH₃)— or —C(CH₃)₂— unit; as well as pharmaceutically acceptable salts and derivatives of said arylalkyl- or aryloxyalkyl-substituted oxirane carboxylic acid: Preferred derivatives are the alkyl esters of the arylalky- and aryloxyalkyl-substituted oxirane carboxylic acids, especially the ethyl esters.

Particularly useful compounds for the inhibition of CPT-1 and falling under formula I above are 2-(6-(4-chlorophenoxy)hexyl)oxirane-2-carboxylic acid ethyl ester (Etomoxir), 2-(6-(4-difluoromethoxyphenoxy) hexyl)oxirane-2-carboxylic acid ethyl ester, 2-(5-(4-difluoromethoxyphenoxy) pentyl)-oxirane-2-carboxylic acid ethyl ester, and 2-(5-(4-acetylphenoxy) pentyl)-oxirane-2-carboxylic acid ethyl ester, Etomoxir being especially preferred.

Other useful CPT-1 inhibitors are sodium-2-(5-(4-chlorophenyl)pentyl)-oxirane-2-caboxylate (Clomoxir), Perhexiline, Trimetazidine, sodium-4-hydroxyphenylglycine (Oxfenicine), 2-tetradecylglycidate (TDGA), and derivatives thereof.

Furthermore, CPT-1 inhibition can be achieved by use of a factor which increases the level of Malonyl-CoA, since Malonyl-CoA is a physiologic inhibitor of CPT-1. Suitable factors for increasing the Malonyl-CoA level can be selected from the group consisting of an activator of the Acetyl-CoA-Carboxylase or an activator of the Citrate-Synthase or an inhibitor of the AMP-Kinase, an inhibitor of the Fatty Acid Synthase or an inhibitor of the Malonyl-CoA-Decarboxylase.

Another option to decrease the fatty acid oxidation is to inhibit the fatty acid binding protein(s) (FABP) which is/are responsible for the binding and transportation of free fatty acids through the cytoplasm of a cell to the mitochondria. A suitable inhibitor (“surrogate inhibitor”) can be a structure which mimics a fatty acid. Examples for structures which mimic fatty acids are fluorescent fatty acid derivatives. As a first surrogate inhibitor, cis-parinaric acid (cPA) can be noted which has been reported for measurement of ligand binding affinities of different FABPs (see e.g. Sha, R. S. et al., “Modulation of ligand binding affinity of the adipocyte lipid-binding protein by selective mutation. Analysis in vitro and in situ”, J. Biol. Chem. 1993, 7885-7892). A second surrogate inhibitor is 12-(anthroyloxy)-oleic acid (12-AO) (see also Sha, R. S. ct al., 1993, cited above). A third surrogate inhibitor is 8-anilino-naphthalene-1-sulfonic acid (ANS). ANS has been described in the context of a displacement assay with FABPs (see e.g. Kane C. D. et al., “A simple assay for intracellular lipid-binding proteins using displacement of 1-anilinonaphthalene 8-sulfonic acid”, Anal. Biochem. 1996, 197-204), and a structure of A-FABP in complex with ANS has been published (see Ory J. J. et al., “Studies of ligand binding reaction of adipocyte lipid binding protein using the fluorescent probe 1,8-anilinonaphlhalene-8-sulfonate”, Biophys. J. 1999, 1107-1116).

In addition, the present invention comprises a method for the inhibition of the expression and/or activity of any enzyme involved in the fatty acid oxidation. Such enzymes are, besides the above-mentioned CPT-1, preferably selected from the group consisting of Phospholipase A, Lipoproteinlipase, Hormone sensitive Lipase, Monoacylglycerol-Lipase, Acyl-CoA-Synthetase, Camitin-Acylcarnitin-Translocase, Carnitin-Palmitoyl-Transferase-2 (CPT-2), Acyl-CoA-Dehydrogenase, Enoyl-CoA-Hydratase, L-3-Hydroxyacyl-CoA-Dehydrogenase, or Thiolase.

Furthermore, the fatty acid oxidation can be inhibited by use of an antisense oligonucleotide or a dominant negative mutant of any enzyme involved in the fatty acid oxidation, particularly the enzymes CPT-1, Acetyl-CoA-Carboxylase, Phospholipase A, Lipoproteinlipase, Hormone sensitive Lipase, Monoacyl glycerol-Lipase, Acyl-CoA-Synthetase, Camitin-Acylcarnitin-Translocase, CPT-2, Acyl-CoA-Dehydrogenase, Enoyl-CoA-Hydratase, L-3-Hydroxyacyl-CoA-Dehydrogenase, or Thiolase. Besides antisense oligonucleotides and dominant negative mutants of any enzyme involved in the fatty acid oxidation, also ribozymes and dsRNA can be used to inhibit fatty acid oxidation.

Moreover, the present invention relates to the use of at least one agent inhibiting the fatty acid oxidation in an organism for the preparation of a pharmaceutical composition for the prophylaxis and/or treatment of a disease related to mitochondrial dysfunction. Preferably, the organism is a human being. Particularly, the agent inhibiting the fatty acid oxidation is a compound which affects the expression and/or activity of CPT-1. Respective compounds, which are suitable for this purpose, are the arylalkyl- or aryloxyalkyl-substituted oxirane carboxylic acids of the formula land the derivatives thereof set forth above, particularly 2-6-(4-chlorophenoxy) hexyp-oxirane-2-carboxylic acid ethyl ester (Etomoxir), 2-(6-(4-difluoromethoxyphenoxy) hexyl)-oxirane-2-carboxylic acid ethyl ester, 2-(5-(4-difluoromethoxyphenoxy) pentyl)-oxirane-2-carboxylic acid ethyl ester, and 2-(5-(4-acetylphenoxy) pentyl)-oxirane-2-carboxylic acid ethyl ester, Etomoxir being especially to preferred.

Other compounds that can be used in compositions to inhibit the activity and/or expression of CPT-1 are sodium-2-(5-(4-chlorophenyl)pentyl)oxirane-2-caboxylate (POCA), sodium-2-(5-(4-chlorophenyl) pentyl)oxirane-2-caboxylate (Clomoxir), Perhexiline, Trimetazidine, sodium-4-hydroxyphenylglycine (Oxfenicine), 2-tetradecylglycidate (TDGA), and derivatives thereof.

Further compounds that are useful for the inhibition of the expression and/or activity of CPT-1 are compounds which increase the level of Malonyl-CoA, since, as already outlined above, Malonyl-CoA is a physiologic inhibitor of CPT-1. Suitable factors for increasing the Malonyl-CoA level can be selected from the group consisting of an activator of the Acetyl-CoA-Carboxylase or the Citrate-Synthase or an inhibitor of the AMP-Kinase or Malonyl-CoA-Decarboxylase, as well as other factors increasing the level of Malonyl-CoA in cells.

In another preferred embodiment the substance used as a fatty acid oxidation inhibitor is a compound which acts on the expression and/or activity of FABP like compounds having structures which mimic fatty acids. Examples for structures which mimic fatty acids are fluorescent fatty acid derivatives (so-called “surrogate inhibitors”, see above). As a first surrogate inhibitor, cis-parinaric acid (cPA) can be noted which has been reported for measurement of ligand binding affinities of different FABPs (see e.g. Sha, R. S. et al., 1993, cited above). A second surrogate inhibitor is 12-(anthroyloxy)-oleic acid (12-AO) (see also Sha, R. S. et al., 1993, cited above). A third surrogate inhibitor is 8-anilino-naphthalene-1-sulfonic acid (ANS). ANS has been described in the context of a displacement assay with FABPs (see Kane C. D. et al., 1996), and a structure of A-FABP in complex with ANS has been published (see Ory J. J. et al., 1999).

A further embodiment of the present invention comprises the use of inhibitors which are preferably selected from the group consisting of antisense oligonucleotides or dominant negative mutants of any of the above-mentioned enzymes which is involved in fatty acid oxidation.

Because of possible synergistic effects of several fatty acid oxidation inhibitors, the present to invention further comprises the simultaneous use of two or more fatty acid oxidation inhibitors for the preparation of a pharmaceutical composition for the prophylaxis and/or treatment of diseases related to mitochondrial dysfunction. Particularly, effective combinations of fatty acid oxidation inhibitors can be the simultaneous use of a CPT-1 inhibitor and a FABP inhibitor, or the simultaneous use of a CPT-1 and CPT-2 inhibitor.

The active compounds (inhibitors or inhibiting agents) according to the present invention are either used as such, or preferably in combination with one or more suitable adjuvant(s) and/or one or more pharmaceutically active and/or acceptable carrier(s), exipient(s), diluent(s), filler(s), binder(s), disintegrant(s), lubricant(s), glident(s), coloring agent(s), flavoring agent(s), opaquing agent(s) and plasticizer(s). The administrable form of the pharmaceutical composition is not limited to a specific route. Routes of administration of the compositions according to the present invention to an individual include but are not limited to inhalation, oral and parenteral, including dermal, intradermal, intragastral, intracutan, intravasal, intravenous, intramuscular, intraperitoneal, intranasal, intravaginal, intrabucal, percutan, rectal, subcutaneous, sublingual, topical or transdermal application. Suitable forms for oral administration are pills, tablets, film tablets, dragées (coated tablets), capsules, powders, emulsions, suspensions or solutions. A suitable form for non-oral administration are e.g. suppositories. A particularly preferred embodiment is the use of active compounds in combination with middle chain triglycerides encapsulated in soft gelatine capsules. Administration to an individual may be in a single dose or in repeated doses. Pharmaceutically acceptable salt forms of active compounds and standard pharmaceutical formulation techniques are well known to persons skilled in the art.

Furthermore, the present invention relates to a pharmaceutical composition comprising at least one agent inhibiting fatty acid oxidation. The agents inhibiting fatty acid oxidation are those which are described in more detail above.

Diseases related to mitochondrial dysfunction are, for example, Morbus Alzheimer, Morbus Huntington, Morbus Parkinson, amyotrophic lateral sclerosis, inflammatory diseases, acute traumatic events such as surgery or injury, AIDS related wasting due to the toxicity of reverse transcriptase inhibitors, mitochondrial myopathies, senescence and ageing, neuronal ischemia, a polyglutamine disease, dystonia, Leber's heredity optic neuropathy (LHON), schizophrenia, to stroke, myodegenerative disorders, Mitochondrial Encephalomyopathy Lactic Acidosis and Strokelike Episodes (MELAS), Myoclonic Epilepsy associated with Ragged-Red Fibers (MERRF), Neuropathy, Ataxia, and Retinitis Pigmentosa (NARP), Progressive External Ophthalmoplegia (PEO), Leigh's disease, Kearns-Sayres Syndromes, muscular dystrophy, myotonic distrophy, chronic fatigue syndrome, Friedreich's Ataxia; developmental delay in cognitive, motor, language, executive function or social skills; epilepsy, peripheral neuropathy, optic neuropathy, autonomic neuropathy, neurogenic bowel dysfunction, sensorineural deafness, neurogenic bladder dysfunction, migraine; renal tubular acidosis, hepatic failure, lactic acidemia, parodontosis, Duchenne muscular dystrophy, Becker's muscular dystrophy, McArdle's disease, abnormities of testosterone synthesis and/or hypoparathyroidism. The cause for mitochondrial dysfunction can be inherited, sporadic or induced by the environment. According to the present invention, prophylaxis against and/or treatment of these diseases can be effected by the methods, compositions and uses described in the present application.

Furthermore, the present invention refers to a method to investigate the effect of fatty acid oxidation inhibitors on mitochondrial function in vitro. This method comprises the cultivation of cells under conditions which are essential for the survival of mitochondria. Under these conditions, different agents which damage mitochondria are used to introduce mitochondrial dysfunction in the absence and/or presence of at least one fatty acid oxidation inhibitor. The mitochondrial function is then monitored. Specifically, the method comprises the following steps:
a) cultivating cells under conditions which are essential for mitochondrial survival;
b) adding at least one mitochondria damaging agent to induce mitochondrial dysfunction;
c) adding at least one fatty acid oxidation inhibitor; and
d) monitoring the mitochondrial function.

Suitable agents to damage mitochondria are leukotoxin, UVB (280-320 nm), UCN-01 (7-hydroxystaurosporine), 1-beta-Darabinofuranosylcrosine, PD184352, PD98059, or U0126 and/or oxidative stress reagents, like H202.

Examples for the cells that can be investigated are neuronal cells (like e.g. PC12 cells), heart cells (like e.g. primary cardiomyocytes, C2C12 cells or H9C2 cells), or cancer cells (like e.g. U937, HL-60, Jurkat or HeLa cells).

Mitochondrial function can be monitored by a variety of methods (e.g. cytochrome c release, respiratory activity, ATP production, measuring the mitochondrial membrane potential, measuring the activity of caspase 3, measuring mitochondrial damage, measuring DNA fragmentation, terminal uridine nick end-labeling assay (TUNEL assay), measuring the is growth and survival of cells by counting cell numbers) in the absence or presence of different fatty acid oxidation inhibitors. The release of cytochrome c can e.g. be measured by Western blot or immunefluorescence assay, and the ATP production can e.g. be measured by the ATP level in one or more cell(s).

1. A method for preventing and/or treating a disease related to mitochondrial dysfunction by inhibiting the fatty acid oxidation of one or more cells of an organism.

2. The method according to claim 1, wherein the organism is a human.

3. The method according to claim 1 or 2, wherein the fatty acid oxidation is inhibited by inhibiting the expression and/or activity of the enzyme Carnitin-Palmitoyl-Transferase-1 (CPT-1).

4. The method according to claim 3, wherein said CPT-1 inhibition is achieved by means is of at least one arylalkyl- or aryloxyalkyl-substituted oxirane carboxylic acid of the following formula I 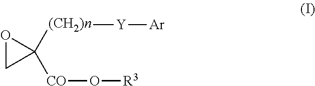
wherein
Ar is a substituted phenyl radical 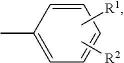
a 1- or 2-naphthyl radical which is substituted by a radical R⁴, or a heterocyclic radical Het;
R¹ is a hydrogen atom, a halogen atom, a 1-4 C lower alkyl group, a 1-4 C lower alkoxy group, a nitro group or a trifluoromethyl group;
R² is one of the groups 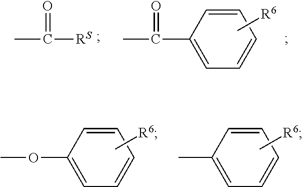
or a fully or predominantly fluorine-substituted 1-3 C alkoxy group or has one of the meanings of R¹;
R³ is a hydrogen atom or a 1-4 C lower alkyl group;
R⁴ is a hydrogen atom, a 1-4 C lower alkyl group, an optionally fully or predominantly fluorine-substituted 1-3 C alkoxy group, or a halogen atom;
R³ is a 1-4 C lower alkyl group;
R⁶ is a hydrogen atom, a halogen atom, or a 1-4 C lower alkyl group;
Y is the grouping —O— or —CH₂—;
n is an integer from 2 to 8; and
Het is a heterocyclic ring, which preferably has 5 members and is selected from the group consisting of thiophene, thiazole, isothiazole, pyrrole, and, particularly preferably, pyrazole, and which may carry 1 or 2 identical or different substituents R¹,
whereby the chain —(CH₂)— may optionally be interrupted by a —CH(CH₃)— or —C(CH₃)₂— unit,
as well as pharmaceutically acceptable salts and derivatives of said arylalkyl- or aryloxyalkyl-substituted oxirane carboxylic acid.

5. The method according to claim 4, wherein said arylalkyl- or aryloxyalkyl-substituted oxirane carboxylic acid of formula I is 2-(6-(4-chlorophenoxy)hexyl)oxirane-2-carboxylic acid ethyl ester (Etomoxir), 2-(6-(4-difluoromethoxyphenoxy)hexyl) oxirane-2-carboxylic acid ethyl ester, 2-(5-(4-difluoromethoxyphenoxy)pentyl) oxirane-2-carboxylic acid ethyl ester, or 2-(5-(4-acetylphenoxy)pentyloxirane-2-carboxylic acid ethyl ester.

6. The method according to claim 3, wherein said CPT-1 inhibition is achieved by the use of sodium-2(5-(4-chlorophenyl)pentyl-oxirane-2-caboxylate (Clomoxir), Perhexiline, Trimetazidine, sodium-4-hydroxyphenylglycine (Oxfenicine), 2-tetradecylglycidate (TDGA), and derivatives thereof.

7. The method according to claim 3, wherein said CPT-1 inhibition is achieved by the use of a factor which increases the Malonyl-CoA-level.

8. The method according to claim 7, wherein the factor which increases said Malonyl. CoA-level is an activator of the Acetyl-CoA-Carboxylase, an activator of the Citrate. Synthase, an inhibitor of the AMP-Kinase, an inhibitor of the Fatty Acid Synthase or an inhibitor of the Malonyl-CoA-Decarboxylase.

9. The method according to claim 1 or 2, wherein said fatty acid oxidation is inhibited by inhibiting the expression and/or activity of fatty acid binding protein (FABP).

10. The method according to claim 9, wherein said inhibiting of the expression and/or to activity of FABP is achieved by means of structures which mimic fatty acids.

11. The method according to claim 10, wherein said structures which mimic fatty acids are selected from the group consisting of cis-parinaric acid (cPA), 12-(anthroyloxy)oleic acid (12-AO), or 8-anilino-naphthalene-1-sulfonic acid (ANS).

12. The method according to claim 1 or 2, wherein said fatty acid oxidation is inhibited by inhibiting the expression and/or activity of Phospholipase A, Lipoproteinlipase, Hormone sensitive Lipase, Monoacylglycerol-Lipase, Acyl-CoA-Synthetase, Carnitin-Acylcamitin-Translocase; Camitin-Palmitoyl-Transferase-2 (CPT-2), Acyl-CoA-Dehydrogenase, Enoyl-CoA-Hydratase, L-3-Hydroxyacyl-CoA-Dehydrogenase, or Thiolase.

13. The method according to claim 1 or 2, wherein said inhibition in fatty acid oxidation is achieved by the use of an antisense oligonucleotide or a dominant negative mutant of at least one of the enzymes CPT-1, Acetyl-CoA-Carboxylase, Phospholipase A, Lipoproteinlipase, Hormone sensitive Lipase, Monoacylglycerol-Lipase, Acyl-CoA-Synthetase, Camitin-Acylcarnitin-Translocase, CPT-2, Acyl-CoA-Dehydrogenase, Enoyl-CoA-Hydratase, L-3-Hydroxyacyl-CoA-Dehydrogenase, or Thiolase.

14. The method according to claim 1 or 2, wherein said inhibition in fatty acid oxidation is achieved by the use of ribozymes or dsRNA.

15. The method according to one of the preceding claims, wherein the disease related to mitochondrial dysfunction is Morbus Alzheimer, Morbus Parkinson, amyotrophic lateral sclerosis, inflammatory diseases, acute traumatic events such as surgery or injury, AIDS related wasting due to the toxicity of reverse transcriptase inhibitors, mitochondrial myopathies, senescence and ageing, neuronal ischemia, a polyglutamine disease, dystonia, Leber's heredity optic neuropathy (LHON), schizophrenia, stroke, myodegenerative disorders, Mitochondrial Encephalomyopathy Lactic Acidosis and Strokelike Episodes (MELAS), Myoclonic Epilepsy associated with Ragged-Red Fibers (MERRF), Neuropathy, Ataxia, and Retinitis Pigmentosa (NARP), Progressive External Ophthalmoplegia (PEO), Leigh's disease, Kearns-Sayres Syndromes, muscular dystrophy, myotonic distrophy, chronic fatigue syndrome, Friedreich's Ataxia; developmental delay in cognitive, motor, language, executive function or social skills; epilepsy, peripheral neuropathy, optic neuropathy, autonomic neuropathy, neurogenic bowel dysfunction, sensorineural deafness, neurogenic bladder dysfunction, migraine; renal tubular acidosis, hepatic failure, lactic acidemia, parodontosis, Duchenne muscular dystrophy, Becker's muscular dystrophy, McArdle's disease, abnormities of the testosterone synthesis and/or hypoparathyroidism.

16. Use of at least one agent inhibiting the fatty acid oxidation for the preparation of a pharmaceutical composition for the prophylaxis and/or treatment of a disease related to mitochondrial dysfunction.

17. Use according to claim 16, wherein the organism is a human.

18. Use according to claim 16 or 17, wherein said agent inhibiting the fatty acid oxidation is as defined in one of claims 3 to 14.

19. Use according to one of claims 16 to 18, wherein the disease related to mitochondrial dysfunction is as defined in claim 15.

20. A method to investigate the effect of fatty acid oxidation inhibitors on mitochondrial function in vitro, said method comprising the steps of
a) cultivating cells under conditions which are essential for mitochondrial survival,
b) adding of at least one mitochondria damaging agent to induce mitochondrial dysfunction,
c) adding at least one fatty acid oxidation inhibitor, and
d) monitoring of the mitochondrial function.

21. The method according to claim 20, wherein said cells are selected from the group consisting of neuronal cells, heart cells, or cancer cells.

22. The method according to claim 21, wherein the neuronal cells are PC12 cells, the heart to cells are primary cardiomyocytes, C2C12 cells or H9C2 cells, and the cancer cells are U937, HL-60, Jurkat or HeLa cells.

23. The method according to one of claims 20 to 22, wherein said at least one mitochondria damaging agent is selected from the group consisting of leukotoxin, UVB (280-320 nm), UCN-01 (7-hydroxystaurosporine), 1-beta-Darabinofuranosylcytosine, PD184352, PD98059, or U0126 and/or oxidative stress reagents.

24. The method according to claim 23, wherein the oxidative stress reagent is H₂O₂.

25. The method according to one of claims 20 to 24, wherein a combination of two or more mitochondria damaging agents is administered to the cultivated cells.

26. The method according to claim 25, wherein said combination comprises UCN-01 and PD184352.

27. The method according to one of claims 20 to 26, wherein the fatty acid oxidation inhibitors are added to the cultivated cells prior to the mitochondria damaging agents.

28. The method according to one of claims 20 to 36, wherein the fatty acid oxidation inhibitors are added to the cultivated cells after the mitochondria damaging agents.

29. The method according to one of claims 20 to 28, wherein the fatty acid oxidation inhibitors are added to the cultivated cells at certain points of time during the damaging treatment of the cells.

30. The method according to claim 29, wherein said points of time are from minutes to hours.

31. The method according to one of claims 20 to 30, wherein the incubation time of the fatty acid oxidation inhibitors that are added to the cultivated cells varies from minutes to days.

32. The method according to one of claims 20 to 31, wherein the mitochondrial function is monitored by the release of cytochrome c.

33. The method according to claim 32, wherein the release of cytochrome c is measured by Western blot or by immunefluorescence assays.

34. The method according to one of claims 20 to 33, wherein the mitochondrial function is monitored by ATP production.

35. The method of claim 34, wherein the ATP production is assessed by direct measurement of the ATP level in the cell.

36. The method according to one of claims 20 to 35, wherein the mitochondrial function is monitored by measuring the mitochondrial membrane potential.

37. The method according to one of claims 20 to 36, wherein the mitochondrial function is monitored by measuring the activity of caspase 3.

38. The method according to one of claims 20 to 37, wherein the mitochondrial function is monitored by measuring mitochondrial damage.

39. The method according to one of claims 20 to 38, wherein the mitochondrial function is monitored by measuring DNA fragmentation.

40. The method according to one of claims 20 to 39, wherein the mitochondrial function is monitored by terminal uridine nick end-labeling (TUNEL) assays.

41. The method according to one of claims 20 to 40, wherein the mitochondrial function is monitored by measuring the growth and survival of cells by counting cell numbers.

42. A pharmaceutical composition for the prophylaxis and/or treatment of diseases related to mitochondrial dysfunction, the composition comprising at least one agent inhibiting to the fatty acid oxidation of an organism.

43. The pharmaceutical composition according to claim 42, wherein the agent inhibiting the fatty acid oxidation of an organism is as defined in one of claims 3 to 14.
